(19) United States
(12) Patent Application Publication
(10) Pub. No.: US 2016/0051491 A1
(43) Pub. Date: Feb. 25, 2016

(54) NOVEL USE OF PINOSYLVIN FOR ANGIOGENESIS PROMOTION

(71) Applicant: INDUSTRY-ACADEMIC COOPERATION FOUNDATION, DANKOOK UNIVERSITY, Yongin-si, Gyeonggi-do (KR)

(72) Inventors: Heon Yong PARK, Yongin-si, Gyeonggi-do (KR); Jae Ho PYEE, Gunpo-si, Gyeonggi-do (KR); Hye Rim LEE, Suwon-si, Gyeonggi-do (KR); Eun Sil JEONG, Anyang-si, Gyeonggi-do (KR)

(73) Assignee: INDUSTRY-ACADEMIC COOPERATION FOUNDATION, DANKOOK UNIVERSITY, Yongin-si, Gyeonggi-do (KR)

(21) Appl. No.: 14/768,110

(22) PCT Filed: Mar. 13, 2013
(86) PCT No.: PCT/KR2013/002029

§ 371 Date: Nov. 5, 2015

(30) Foreign Application Priority Data
Feb. 15, 2013 (KR) 10-2013-0016623

(51) Int. Cl. A61K 31/05

(52) U.S. Cl. CPC A61K 31/05

(57) ABSTRACT

The present invention relates to a pharmaceutical composition for preventing or treating angiogenesis-dependent diseases containing pinosylvin or a pharmaceutically acceptable salt thereof as an active ingredient, a food composition for preventing or improving angiogenesis-dependent diseases containing pinosylvin or a pharmaceutically acceptable salt thereof as an active ingredient, a method for treating angiogenesis-dependent diseases including administering the pharmaceutical composition to a subject suspected of having the angiogenesis-dependent diseases, and a method for promoting angiogenesis including administering pinosylvin or a pharmaceutically acceptable salt thereof to a subject in need of angiogenesis promotion.

### BACKGROUND OF THE INVENTION

1. Field of the Invention

The present invention relates to a pharmaceutical composition for preventing or treating angiogenesis-dependent diseases containing pinosylvin or a pharmaceutically acceptable salt thereof as an active ingredient, a food composition for preventing or improving angiogenesis-dependent diseases containing pinosylvin or a pharmaceutically acceptable salt thereof as an active ingredient, a method for treating angiogenesis-dependent diseases including administering the pharmaceutical composition to a subject suspected of having the angiogenesis-dependent diseases, and a method for promoting angiogenesis including administering pinosylvin or a pharmaceutically acceptable salt thereof to a subject in need of angiogenesis promotion.

2. Description of the Related Art

Pine trees are distributed around the world, especially in Eastern Asia including Korea, where pine needles and pollen products have been used in traditional oriental medicine for the preservation of health. Pycnogenol is a well-known extract of pine bark of Pinus pinaster Ait. Subsp. atlantica. In vitro and in vivo studies of pycnogenol revealed that pycnogenol can act as an anti-oxidant, and in vascular biology, pycnogenol is known to play a beneficial role in chronic venous insufficiency and hypertension. Based on these cardiovascular activities of pycnogenol, it has been suggested that extracts of pine trees may be used in alternative medicine or in the nutraceutical field. However, the functional compounds of pine tree extracts remain largely unknown.

Pinosylvin is a naturally-occurring stilbenoid which is present in pine wood and is involved in decay resistance of heartwood. Due to its antibacterial and antifungal activities, pinosylvin is suggested as the functional compound of pine trees. However, the potential medicinal benefits of pinosylvin other than as a functional compound have not been well studied. Pinosylvin and a series of its derivatives were previously synthesized, and their inhibitory effects on prostaglandin E2 production in lipopolysaccharide (LPS)-induced mouse macrophage cells were reported (Park et al., Bioorg Med Chem Lett. 2004 Dec. 6; 14(23):5895-8), and also there was a report on their antibacterial and antifungal activities (Lee et al., Fitoterapia. 2005 March; 76(2):258-60). However, little is known about the effect of angiogenesis promotion by pinosylvin.

Angiogenesis is a biological process through which new blood vessels are provided to tissues and organs. Specifically, angiogenesis refers to a process forming new capillary blood vessels from the existing microvessels, and is a fundamental process for forming blood vessels in the body. The physiological angiogenesis normally observed in the human body occurs in very limited circumstances such as development of embryos and fetuses, growth of uterus, proliferation of placenta, formation of corpus luteum, and healing of wounds. Even during such periods, the angiogenesis process is strictly regulated and stops once the necessary role is accomplished. Angiogenesis is under the strict control of angiogenic factors, and reportedly, the phenotypes of the angiogenic factors can be changed by the overall balance between up-regulation of angiogenesis-stimulating factors and down-regulation of angiogenesis-inhibiting factors.

The process of forming new blood vessels is highly complex and sophisticated, and may be summarized as shown below. First, when a stimulus for angiogenesis reaches the existing blood vessels, it causes them to become swollen and their membrane permeability to increase. Second, fibrins become released from the swollen blood vessels and deposited on the cytoplasmic matrix of the neighboring blood vessels. Third, enzymes that can decompose basal membranes of the existing blood vessels are activated, and endothelial cells therein become released through the decomposed basal membranes, proliferate in the matrices of the neighboring cells, and migrate thereon. Lastly, the endothelial cells lined-up in a line establish vascular vessels, thereby forming new blood vessels.

When there is an excess formation of new blood vessels, it may aggravate diseases such as cancer, however, the promotion of angiogenesis can be used for the treatment of particular diseases. For example, angiogenesis is essentially required for wound healing and tissue regeneration. This is because newly formed blood vessels can assist live cells by supplying nutrients thereto, promote the formation of granulation tissues, and promote removal of debris. For these reasons, materials capable of promoting angiogenesis can be used for wound healing and tissue regeneration. Additionally, since diseases, such as atherosclerosis, myocardial infarction, and angina pectoris, can occur due to abnormal blood supply, the materials capable of promoting angiogenesis may be used for treating these diseases.

### SUMMARY OF THE INVENTION

The present inventors, while endeavoring to develop a novel material capable of promoting angiogenesis, discovered that pinosylvin can inhibit apoptosis of vascular endothelial cells, promote proliferation thereof, and promote angiogenesis, and thus confirmed that pinosylvin can be used for treating diseases requiring angiogenesis promotion, for example, myocardial infarction, atherosclerosis, etc., thereby completing the present invention.

An object of the present invention is to provide a pharmaceutical composition for preventing or treating angiogenesis-dependent diseases containing pinosylvin or a pharmaceutically acceptable salt thereof as an active ingredient.

Another object of the present invention is to provide a food composition for preventing or improving angiogenesis-dependent diseases containing pinosylvin or a pharmaceutically acceptable salt thereof as an active ingredient.

Still another object of the present invention is to provide a composition for promoting angiogenesis containing pinosylvin or a pharmaceutically acceptable salt thereof as an active ingredient.

Still another object of the present invention is to provide a method for treating angiogenesis-dependent diseases including administering pinosylvin or a pharmaceutically acceptable salt thereof to a subject suspected of having the angiogenesis-dependent disease(s).

Still another object of the present invention is to provide a method for promoting angiogenesis including administering pinosylvin or a pharmaceutically acceptable salt thereof to a subject in need of angiogenesis promotion.

### Advantageous Effects of Invention

Since pinosylvin possesses an angiogenesis-promoting activity, it can be effectively used in the field requiring angiogenesis promotion, e.g., for the treatment of angiogenesis-dependent diseases, etc.

### BRIEF DESCRIPTION OF THE DRAWINGS

FIG. 1 shows the results of an anti-apoptotic effect of pinosylvin on endothelial cells. (A) Confluent BAECs were cultured in a serum-starved medium for more than 12 hours, and the cells were incubated for an additional 72 hours at the concentration of pinosylvin (PIN) indicated in FIG. 1A. Top panel: arrows indicate dead cells; Bottom panel: dead cells were quantified and plotted as bar graphs (means±S.E., n=3). *P<0.05. (B) Confluent endothelial cells were pretreated with a vehicle or 1 pM pinosylvin in DMEM containing 10% serum and then incubated with 100 mM etoposide (ETO) for 18 hours. Apoptotic cells (round and shrunken cells) were observed and counted under a microscope, and the fragmented nuclei were detected by Hoechst 33258 staining. Arrows indicate the fragmented nuclei, and the bar graphs represent the percentages of apoptotic cells (means±S.E., n=4). *P<0.05. (C) Serum-depleted cells (grown in 0.5% FBS-DMEM) were stimulated with 1 pM pinosylvin or 100 mM ETO. Then, the cells were lysed, and the proteins in the cell lysates were resolved by SDS-PAGE, transferred to PVDF membranes and immunoblotted with anti-caspase-3 antibodies. (D) The cell lysates were reacted in a reaction buffer containing Ac-DEVD-pNA at 37° C. for 2 hours. Caspase-3 activity was determined by measuring the absorbance of the reactions at 405 nm, and the bar graph represents means±S.E. (n=3). **P<0.03, *P<0.05.

FIG. 2 shows the results of activation of Akt and eNOS by pinosylvin. Serum-depleted BAECs were activated by treatment with 1 pM pinosylvin for 0 minutes, 5 minutes, 15 minutes, 30 minutes, or 60 minutes, and the cell lysates were resolved by SDS-PAGE, transferred to PVDF membranes and immunoblotted with the antibodies to the proteins indicated in FIG. 2.

FIG. 3 shows the results of induction of endothelial cell proliferation by pinosylvin. (A) Thirty percent confluent BAECs were incubated with DMEM containing no compounds (vehicle) and 1 pM pinosylvin (Experimental Group), respectively, and the grown cells were observed under a microscope. (B) Live cells grown in DMEM containing none or 20% serum for 24 hours were measured using the WST-1 reagent. The measured results were shown as a bar graph (means±S.E., n=3). *P<0.05. (C) BAECs were pretreated with 2.5 mM L-NAME and then treated with pinosylvin at the concentration of pinosylvin (PIN) indicated in FIG. 3C for 24 hours. The bar graph represents relative live cell amounts detected by the WST-1 assay (means±S.E., n=3). *P<0.05.

FIG. 4 shows the results of stimulation of cell migration and tube formation in endothelial cells by pinosylvin. (A) Confluent endothelial cells were cultured in a serum-starved medium and then scraped with a pipette tip. Cells were then incubated with DMEM containing no pinosylvin, in DMEM containing 1 pM pinosylvin, or DMEM containing both 2.5 mM L-NAME and 1 pM pinosylvin, respectively. After 12 hours, the migrated cells were observed under a microscope. Data for cell migration were represented as a bar graph (means±S.E., n=3). *P<0.05. (B) BAEC suspensions (1.5×10⁴ cells/100 mL) containing DMEM containing no pinosylvin, DMEM containing 1 pM pinosylvin, or DMEM containing both 2.5 mM L-NAME and 1 pM pinosylvin were added to ECM gel, respectively, and then additionally incubated to form endothelial tubes. Then, endothelial tubes were observed under a microscope.

### PREFERRED EMBODIMENTS OF THE INVENTION

In an aspect, the present invention provides a pharmaceutical composition for preventing or treating angiogenesis-dependent diseases containing pinosylvin or a pharmaceutically acceptable salt thereof as an active ingredient.

As used herein, the term “pinosylvin” is a stilbenoid compound, also called 5-[(E)-2-phenylethenyl]benzene-1,3-diol. Pinosylvin has the structure of Formula 1 shown below.

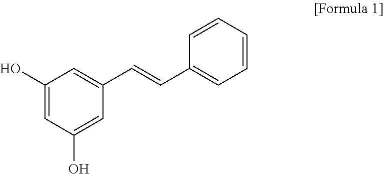 [SMILES: Oc1cc(O)cc(/C=C/c2ccccc2)c1]

For the purposes of the present invention, pinosylvin refers to a compound having the activity of angiogenesis promotion. Pinosylvin may be synthesized by a method of synthesizing compounds known in the art, purchased from among those available on the commercial market, or extracted and separated from heartwood or leaves of Pinacea for use. The antibacterial activity of pinosylvin has been reported. However, the effect of pinosylvin on angiogenesis promotion has not been known and is herein first confirmed by the present inventors.

As used herein, the term “pharmaceutically acceptable salt” refers to a salt which can be pharmacologically used among the salts where anions and cations are bound by electrostatic interaction, and conventionally, a metal salt, a salt with an organic base, a salt with an inorganic acid, a salt with an organic acid, a salt with a basic or acidic amino acid, etc. For example, the metal salt may be an alkali metal salt (a sodium salt, a potassium salt, etc.), an alkali metal salt (a calcium salt, a magnesium salt, a barium salt, etc.), an aluminum salt, etc.; the salt with an organic base may be a salt with triethylamine, pyridine, picoline, 2,6-lutidine, ethanolamine, diethanolamine, triethanolamine, cyclohexylamine, dicyclohexylamine, N,N-dibenzylethylenediamine, etc.; the salt with an inorganic acid may be a salt with hydrochloric acid, hydrobromic acid, nitric acid, sulfuric acid, phosphoric acid, etc.; the salt with an organic acid may be a salt with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid, etc.; the salt with a basic amino acid may be a salt with arginine, lysine, ornithine, etc.; and the salt with an acidic amino acid may be a salt with aspartic acid, glutamic acid, etc.

As used herein, the term “angiogenesis” refers to a process of newly forming blood vessels, i.e., new blood vessels are generated into cells, tissues, or organs. This process of forming new blood vessels may be achieved by i) activating receptors present in the vascular endothelial cells of the existing blood vessel in a resting state by angiogenic growth factors, ii) secreting proteases that decompose the neighboring basal membranes and the cytoplasmic matrices by the activated vascular endothelial cells, and iii) allowing the vascular endothelial cells to escape from the existing vascular walls and migrate toward the tissues, which secrete angiogenic factors, and proliferate.

As used herein, the term “angiogenesis-dependent diseases” refers to diseases which accompany symptoms of an abnormal blood supply or incomplete angiogenesis. The diseases may not be particularly limited as long as they can be prevented or treated by the angiogenesis promotion via pinosylvin or a pharmaceutically acceptable salt thereof of the present invention, and may include wounds, burns, atherosclerosis, myocardial infarction, angina pectoris, varices, cerebral vascular dementia, etc. The angiogenesis-dependent diseases require the promotion of angiogenesis in preventing and treating the diseases. For example, in the case of wounds and burns, angiogenesis is required to supply nutrients for the recovery of the damaged tissues, and in the case of atherosclerosis, myocardial infarction, angina pectoris, varices, cerebral vascular dementia, angiogenesis may be required for the improvement of blood flow disorder. The composition of the present invention having the activity of promoting angiogenesis containing pinosylvin or a pharmaceutically acceptable salt thereof may be effectively used for the prevention or treatment of angiogenesis-dependent diseases.

As used herein, the term “prevention” refers to any action resulting in suppression or delay of the onset of angiogenesis-dependent diseases by the administration of the pharmaceutical composition according to the present invention, and the term “treatment” refers to any action resulting in improvement in symptoms of angiogenesis-dependent diseases or the beneficial alteration by the administration of the composition according to the present invention.

Additionally, the pharmaceutical composition of the present invention containing pinosylvin or a pharmaceutically acceptable salt thereof may further contain an appropriate carrier, excipient, or diluents which are conventionally used in the preparation of pharmaceutical compositions.

The pharmaceutical composition of the present invention may be prepared in any formulation selected from the group consisting of tablets, pills, powders, granules, capsules, suspensions, liquid medicine for internal use, emulsions, syrups, sterile injection solutions, non-aqueous solvents, suspensions, emulsions, lyophilized formulations, and suppositories, and may be prepared in various formulations for oral or parenteral administration. The formulations may be prepared using a commonly used diluent such as a filler, an extender, a binder, a humectant, a disintegrant, a surfactant, etc., or excipient. Solid formulations for oral administration may include tablets, pills, powders, granules, capsules, etc., and these solid formulations may be prepared by adding at least one excipient, e.g., starch, calcium carbonate, sucrose or lactose, gelatin, etc. Additionally, a lubricant such as magnesium stearate, talc, etc., may be used, in addition to the simple excipient. Liquid formulations for oral administration may include suspensions, liquid medicine for internal use, emulsions, syrups, etc., and various excipients such as humectants, sweeteners, fragrances, and preservatives, may be used, in addition to the simple diluents such as water and liquid paraffin. Formulations for parenteral administration may include sterile aqueous solutions, non-aqueous solvents, suspensions, emulsions, lyophilized formulations, suppositories, etc. Examples of the non-aqueous solvents and suspensions may include a vegetable oil such as propylene glycol, polyethylene glycol, and olive oil, an injectable ester such as ethyl oleate, etc. Examples of bases for suppositories may include Witepsol, macrogol, Tween 61, cacao butter, laurinum, glycerogelatin, etc.

The composition of the present invention is administered in a pharmaceutically effective amount. As used herein, the term “pharmaceutically effective amount” refers to an amount sufficient for the treatment of diseases at a reasonable benefit/risk ratio applicable to a medical treatment without causing any adverse effects, and the level of the effective dose may be determined based on the factors including the kind of subject, severity of illness, age, sex, kind of disease, drug activity, drug sensitivity, administration time, administration route and dissolution rate, length of treatment, factors including drug(s) to be concurrently used in combination, and other factors well known in the medical field. The composition of the present invention may be administered as an individual therapeutic agent, in combination with other therapeutic agents, or sequentially or simultaneously with a conventional therapeutic agent(s), and may be administered once or multiple times. It is important to administer an amount to obtain the maximum effect with a minimum amount without adverse effects, considering the factors described above, and these factors can easily be determined by one of ordinary skill in the art.

A preferable dose of the composition of the present invention may vary depending on the health conditions and body weight of a patient, severity of illness, drug type, administration route, and duration. The administration may be performed once daily or in a few doses. The composition may be administered to various mammals including mice, cattle, humans, etc., via various administration routes. The administration method may include any conventional method in the art, without limitation, e.g., oral, rectal or intravenous, intramuscular, subcutaneous, intrauterine epidural, or intracerebrovascular injection.

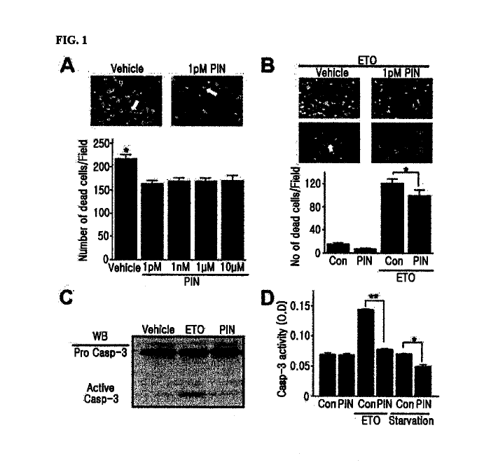

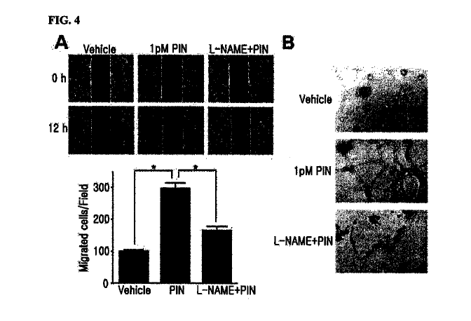

In an exemplary embodiment of the present invention, the effect of pinosylvin on vascular endothelial cells was investigated. As a result, it was confirmed that pinosylvin not only significantly reduced the apoptosis of endothelial cells induced by serum starvation or etoposide but also induced the proliferation of endothelial cells (FIGS. 1 to 3). Furthermore, it was confirmed that pinosylvin, by the activity mediated by eNOS, can promote the cell migration of endothelial cells and induce tube formation, thereby promoting angiogenesis (FIG. 4). Accordingly, the composition containing pinosylvin of the present invention may be effectively used for preventing and treating diseases which require angiogenesis, i.e., angiogenesis-dependent diseases such as wounds, burns, atherosclerosis, angina pectoris, etc.

In another aspect, the present invention provides a food composition for preventing or improving angiogenesis-dependent diseases containing pinosylvin or a pharmaceutically acceptable salt thereof as an active ingredient.

Pinosylvin, the pharmaceutically acceptable salt, and angiogenesis-dependent diseases are the same as described above.

Since pinosylvin possesses the activity of promoting angiogenesis, pinosylvin or a pharmaceutically acceptable salt thereof may be added to a food composition for the purpose of preventing or improving angiogenesis-dependent diseases. When pinosylvin or a pharmaceutically acceptable salt thereof is used as a food additive, it may be added as is or in combination with other food(s) or component(s). Additionally, the amount of its addition may be appropriately determined according to the purposes of its use.

There is no particular limitation on the kinds of foods of the present invention. Examples of the foods in which pinosylvin or a pharmaceutically acceptable salt thereof may be added may include meats, sausages, bread, chocolates, candies, snacks, cookies, pizzas, ramen, other noodles, gums, dairy products including ice cream, various kinds of soups, beverages, teas, drinks, alcoholic beverages, vitamin complexes, etc., and may include all kinds of foods from the conventional point of view, and may also include foods used as animal feeds.

The food composition of the present invention, in addition to the above, may also include various kinds of nutrients, vitamins, electrolytes, flavoring agents, coloring agents, pectic acid and salts thereof, alginic acid and salts thereof, organic acids, protective colloidal thickeners, pH adjusting agents, stabilizers, antiseptics, glycerins, alcohols, carbonated beverages, carbonating agents used in carbonated beverages, etc. Additionally, fruit flesh for preparing natural fruit juices, fruit juices, and vegetable juices may be contained. Additionally, the foods may be prepared in the form of tablets, granules, powders, capsules, liquid solutions, pills, etc., according to the preparation method known in the art. There is no particular limitation in other ingredients except that the present invention contains pinosylvin as an active ingredient, and various flavoring agents or natural carbohydrates may be contained as additional ingredients.

In another aspect, the present invention provides a composition for promoting angiogenesis containing pinosylvin or a pharmaceutically acceptable salt thereof as an active ingredient.

Pinosylvin, the pharmaceutically acceptable salt thereof, and angiogenesis are the same as described above.

Since pinosylvin possesses an angiogenesis-promoting activity, a composition containing pinosylvin or a pharmaceutically acceptable salt thereof may be used as a composition for angiogenesis promotion, i.e., an angiogenesis-promoting agent.

In another aspect, the present invention provides a method for treating angiogenesis-dependent diseases including administering the pharmaceutical composition to a subject suspected of having an angiogenesis-dependent disease.

The pharmaceutical composition, angiogenesis-dependent diseases, and treatment thereof are the same as described above.

Specifically, the treatment method of the present invention includes administering a pharmaceutically effective amount of the pharmaceutical composition to a subject suspected of having an angiogenesis-dependent disease. The subject may refer to entire mammals including dogs, cattle, horses, rabbits, mice, rats, chickens, and humans, but is not limited thereto. The pharmaceutical composition may be administered via parenteral, subcutaneous, intraperitoneal, intrapulmonary, or intranasal administration, and may be administered by an appropriate method including an intralesional administration for topical treatment, if necessary. A preferable amount of the pharmaceutical composition of the present invention may vary depending on the health conditions and weight of a subject, severity of illness, drug type, administration route, and length of treatment, but it may be appropriately selected by one of ordinary skill in the art.

In another aspect, the present invention provides a method for promoting angiogenesis including administering the pinosylvin or a pharmaceutically acceptable salt thereof to a subject in need of angiogenesis promotion.

Pinosylvin, the pharmaceutical composition, and angiogenesis promotion are the same as described above.

Specifically, the treatment method of the present invention includes administering a pharmaceutically effective amount of pinosylvin or a pharmaceutically acceptable salt thereof to a subject in need of angiogenesis promotion. The subject may refer to entire mammals including dogs, cattle, horses, rabbits, mice, rats, chickens, and humans, but are not limited thereto. The pharmaceutical composition may be administered via parenteral, subcutaneous, intraperitoneal, intrapulmonary, or intranasal administration, and may be administered by an appropriate method including an intralesional administration for topical treatment, if necessary. A preferable amount of the pharmaceutical composition of the present invention may vary depending on the health conditions and weight of a subject, severity of illness, drug type, administration route, and length of treatment, but it may be appropriately selected by one of ordinary skill in the art.

### DETAILED DESCRIPTION OF THE INVENTION

Hereinafter, the present invention will be described in more detail with reference to the following Examples. However, these Examples are for illustrative purposes only, and the invention is not intended to be limited by these Examples.

### EXAMPLE 1

### Cell Culture and Drug Treatment

Bovine aortic endothelial cells (BAECs) were obtained from the descending thoracic aortas, and maintained in a growth medium (DMEM (1 g/L glucose, Life technologies, Inc.) containing 20% fetal bovine serum albumin (FBS, Atlanta Biologicals) without antibiotics) at 37° C. in 5% CO₂. The number of cells used for used in this study was between 3 generations and 10 generations, and they were allowed to grow in untreated culture dishes. Before drug treatment, BAECs were grown to confluency in a 5% CO₂ incubator with growth media containing 50 μg/mL penicillin and 50 μg/mL streptomycin. The cells were then starved for between 4 hours and 24 hours in starvation media (DMEM supplemented with 0.5% FBS and 50 μg/mL penicillin and 50 μg/mL streptomycin).

For the apoptotic analysis, the confluent cells were incubated in 0.5% FBS-DMEM containing 0 μM or 100 μM etoposide (ETO, Sigma) or varying amounts of pinosylvin for 36 hours. Since pinosylvin is not commercially available, it was synthesized as described (Park et al., Cell Physiol Biochem. 2011; 27(3-4): 353-62.). In the experiments conducted to determine which signaling molecules were involved in pinosylvin-induced effects, BAECs were pretreated with 2.5 μM L-NAME (an eNOS inhibitor, Cell Signaling), 1 hour before pinosylvin treatment.

### EXAMPLE 2

### Measurement of Apoptosis

Confluent BAECs were starved for more than 4 hours, treated with pinosylvin, and then incubated for an additional period of time (0 hours, 12 hours, 24 hours, and 48 hours) for the evaluation of time-dependent changes in apoptosis. The round and shrunken apoptotic cells were then examined under a microscope to observe the occurrence of apoptosis. For quantification, the present inventors counted the number of apoptotic cells in the same visual field.

For Hoechst staining, the confluent cells were incubated with 0.5% FBS-DMEM containing 100 μM ETO without any compound (vehicle control) for 36 hours. The BAECs were then fixed with Conroy's fixative for 10 minutes and washed with phosphate buffered saline (PBS). The cells were air-dried for 10 minutes. After air-drying, the cells were stained with Hoechst 33258 (12.5 μg/mL, Sigma) for 30 minutes at room temperature. After incubation, the stained cells were thoroughly washed with PBS and the nuclei were observed under a fluorescence microscope (Zeiss Autoplan 2) to confirm the occurrence of apoptosis.

### EXAMPLE 3

### Measurement of Caspase-3 Activity

Caspase-3 activity was measured with Ac-DEVD-pNA as a substrate using the caspase-3 Colorimetric Activity Assay Kit (Chemicon). Specifically, BAECs were grown to confluency in 20% FBS-DMEM and then starved for more than 8 hours. The cells were treated with various apoptotic agents (100 μM ETO) and then lysed. The cell lysates were reacted in a reaction buffer containing 236 μM Ac-DEVD-pNA with or without specific inhibitors (0.1 μM Ac-DEVD-CHO) in 96-well plates at 37° C. for 2 hours. Then, the caspase-3 activity was determined by measuring the absorbance of the reactions at 405 nm using a microplate reader (Bio-Rad, Model 550).

### EXAMPLE 4

### Measurement of Cell Proliferation

Cell proliferation was measured using a cell viability assay kit (Daeil Lab Service Co., LTD., Seoul, Korea). Specifically, confluent BAECs were serum-starved for 16 hours, and then various concentrations of pinosylvin or 20% serum were added and incubated. The cells were washed more than twice with PBS and reacted with the WST-1 reagent. The live cells were then measured at 450 nm using an ELISA microplate reader (Bio-Rad, Model 550).

### EXAMPLE 5

### Western Blotting

The BAECs were grown to confluency in 20% FBS-DMEM, washed with ice-cold PBS, scraped in 250 μL RIPA buffer (50 mM Tris, pH 7.5, 150 mM NaCl, 1% NP-40, 0.5% sodium deoxycholate, and 0.1% SDS), and solubilized for 15 minutes to prepare the cell lysates. All the solubilization procedures were performed at 4° C., and the protein content of the soluble cell lysates was measured using a Bio-Rad DC assay kit (Bio-Rad). The solution lysates (10 μg each) were resolved by SDS-PAGE. The resolved protein bands were then transferred to a polyvinylidene difluoride (PVDF) membrane (Bio-Rad) and probed with p-ERK (cell signaling), p-p38 MAP kinase (cell signaling), p-JNK (cell signaling), p-eNOS (cell signaling), p-Akt (cell signaling) and caspase-3 (cell signaling) for 1 hour to overnight. The membranes were then incubated with a secondary antibody at room temperature for 1 hour, where goat anti-rabbit IgG conjugated to alkaline phosphatase was used as a secondary antibody. Finally, the results were confirmed using a chemiluminescent detection method.

### EXAMPLE 6

### Confirmation of Cell Migration

Confluent BAECs were treated twice with 2 mM thymidine for 18 hours and wounded with a pipette tip. Then, the cells were incubated in the starvation media for 20 hours with increasing amounts of pinosylvin: 1) no pinosylvin, 2) 1 pM pinosylvin, or 3) 1 pM pinosylvin with 2.5 mM L-NAME, in DMEM, respectively. The migrated cells were observed under a microscope and quantified by counting the number of the migrated cells in the wound area.

### EXAMPLE 7

### Tube Formation

ECM gel (Cell Biolabs Inc.) was thawed overnight at 4° C. Then, the thawed gel 50 μL was added into each well of the 96-well plates. To form a gel, the 96-well plates were incubated at 37° C. for 1 hour. Then, the BAEC suspension (1.5×10⁴ cells/100 μL) containing no pinosylvin, 1 pM pinosylvin, or 1 pM pinosylvin with 2.5 mM L-NAME, in DMEM, respectively, was added into a gel and placed in a 5% CO₂ incubator at 37° C. for 12 hours to 18 hours. Then, the endothelial cells were observed under a microscope to confirm tube formation.

### EXPERIMENTAL EXAMPLE 1

### Anti-Apoptotic Effect on Endothelial Cells

In order to examine whether pinosylvin has an anti-apoptotic activity, experiments were performed as shown below.

Specifically, the apoptotic activity was determined based on the number of apoptotic cells, nuclear condensation, and caspase-3 activity, and the results are shown in FIG. 1.

As a result, the control group, where cells were serum-starved for 72 hours, showed that a plurality of cells became round and shrunken, a characteristic of apoptosis, whereas the group where the cells were administered with a low dose of pinosylvin was shown to prevent serum starvation-induced apoptosis (FIG. 1A, top panel). Additionally, cell apoptosis was quantified by counting apoptotic cells per visual field, and as shown in the graphs, the starvation-induced apoptosis was inhibited by 1 pM to 10 μM of pinosylvin (FIG. 1A, bottom panel).

In order to examine whether pinosylvin can inhibit the apoptosis induced by EOT, a different apoptotic agent, the level of apoptosis was measured by Hoechst 33258 staining, and the result is shown in FIG. 1B.

As a result, as shown in FIG. 1B, fragmentation of the nuclei, the characteristic of apoptotic endothelial cells, was detected in cells treated with 100 μM ETO, whereas the cells treated with both 1 pM pinosylvin and 100 μM ETO showed a markedly reduced level (FIG. 1B).

These results suggest the anti-apoptotic effect of pinosylvin.

Next, in order to determine the molecular mechanisms underlying the anti-apoptotic effect of pinosylvin, the cleavage and catalytic activities of caspase-3, which is known to be a major molecule involved in apoptosis, were examined and the results are shown in FIGS. 1C and 1D.

As a result, 100 μM ETO was shown to activate caspase-3, whereas 1 pM pinosylvin inhibited ETO- or starvation-induced caspase-3 activation (FIGS. 1C and 1D). These data suggest that pinosylvin acts as an anti-apoptotic agent in endothelial cells via caspase-3 inactivation.

### EXPERIMENTAL EXAMPLE 2

### Confirmation of eNOS Activation by Pinosylvin

As confirmed in Experimental Example 1, it was suggested that 1 pM pinosylvin can exhibit an anti-apoptotic activity on endothelial cells. In order to determine whether the anti-apoptotic activity of pinosylvin is associated with any of the downstream signal transduction pathways, the effects of 1 pM pinosylvin on the activation of various signaling molecules including eNOS were investigated.

Specifically, it was investigated whether the activation is mediated through ERK, JNK, and p38MAP kinase pathways and/or eNOS and Akt pathways, which are representative signal transduction pathways responsible for apoptosis.

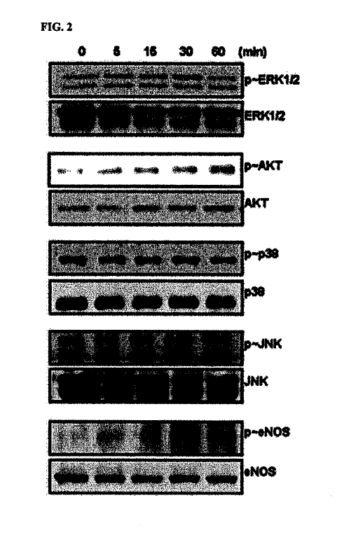

As a result, as shown in FIG. 2, ERK, JNK, and p38 phosphorylation remained largely unchanged, whereas Akt and eNOS phosphorylations were substantially promoted by pinosylvin, thus suggesting that the apoptotic activity of pinosylvin is mediated by Akt and eNOS.

### EXPERIMENTAL EXAMPLE 3

### Confirmation of Endothelial Cell Proliferation by Pinosylvin

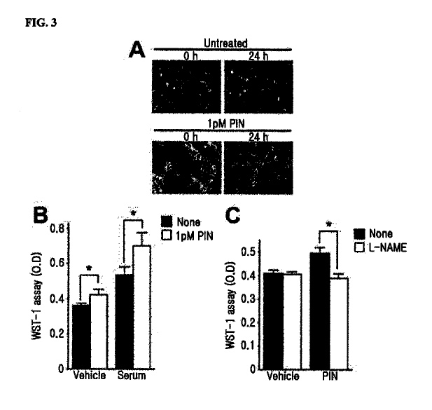

In addition to the anti-apoptotic activity of pinosylvin, the effect of pinosylvin on the proliferation of endothelial cells was examined, and the results are shown in FIG. 3.

As a result, as shown in FIG. 3A, the number of cells increased when treated with 1 pM pinosylvin compared with that of untreated cells, and this indicates the cell proliferative activity of pinosylvin.

Additionally, the cell proliferative activity of pinosylvin was further confirmed by the WST-1 assay, and this cell proliferative activity was observed in the presence and absence of bovine serum, and the results are shown in FIG. 3B. Furthermore, the involvement of eNOS, which was confirmed in Experimental Example 2, was further examined, and the results are shown in FIG. 3C.

As a result, pinosylvin was shown to promote cell proliferation regardless of the presence of serum, and the responsibility of eNOS in this promotion of cell proliferation was confirmed by treating with L-NAME, an eNOS inhibitor (FIGS. 3B and 3C).

### EXPERIMENTAL EXAMPLE 4

### Confirmation of Cell Migration and Tube Formation by Pinosylvin

In addition to the anti-apoptotic effect and cell proliferation promoting effect of pinosylvin, the effect of pinosylvin on the promotion of endothelial cell migration was examined. Since the migration of endothelial cells induces vessel remodeling and becomes a process enabling wound healing, it was examined whether pinosylvin can promote migration of endothelial cells and thus promote angiogenesis, thereby being useful for the treatment of diseases such as wounds, atherosclerosis, etc., which require promotion of angiogenesis.

As a result, as shown in FIG. 4A, pinosylvin was shown to significantly promote cell migration, and that eNOS activity is involved therein.

Additionally, since migration and tube formation of endothelial cells are essential processes in angiogenesis, it was examined whether pinosylvin can promote tube formation along with the migration of endothelial cells, and the results are shown in FIG. 4B.

As shown in FIG. 4B, pinosylvin was shown to significantly increase tube formation compared to the control group. In contrast, when pinosylvin was treated concurrently along with L-NAME, an eNOS inhibitor, the effect of tube formation by pinosylvin was reduced, thus suggesting that the tube formation by pinosylvin can be mediated by eNOS.

Conclusively, these results indicate that the pinosylvin of the present invention has the use of promoting angiogenesis, and thus pinosylvin can be used for the treatment of diseases which require promotion of angiogenesis.

From the foregoing, an ordinary person skilled in the art to which the present invention pertains will be able to understand that the present invention may be embodied in other specific forms without modifying the technical concepts or essential characteristics of the present invention. In this regard, the exemplary embodiments disclosed herein are only for illustrative purposes and should not be construed as limiting the scope of the present invention. On the contrary, the present invention is intended to cover not only the exemplary embodiments but also various alternatives, modifications, equivalents and other embodiments that may be included within the spirit and scope of the present invention as defined by the appended claims.

1. A method for preventing or treating angiogenesis-dependent diseases including administering pinosylvin or a pharmaceutically acceptable salt thereof to a subject suspected of having the angiogenesis-dependent disease.

2. The method of claim 1, wherein the angiogenesis-dependent disease is selected from the group consisting of wounds, burns, atherosclerosis, myocardial infarction, angina pectoris, varices, and cerebral vascular dementia.

3. The method of claim 1, further comprising a pharmaceutically acceptable carrier.

4. The method of claim 1, wherein the composition may be prepared in any of the formulations selected from the group consisting of tablets, pills, powders, granules, capsules, suspensions, liquid medicines for internal use, emulsions, syrups, sterile solutions, non-aqueous solvents, suspensions, emulsions, lyophilized formulations, and suppositories.

5. (canceled)

6. (canceled)

7. A method for promoting angiogenesis including administering pinosylvin or a pharmaceutically acceptable salt thereof to a subject in need of angiogenesis promotion.

8. (canceled)

9. (canceled)
